(19) United States
(12) Patent Application Publication
(10) Pub. No.: US 2024/0100091 A1
(43) Pub. Date: Mar. 28, 2024

(54) METHOD AND KIT FOR TREATING ABNORMAL HOLLOWED SPACE

(71) Applicant: Reddress Ltd., Pardes Hana (IL)

(72) Inventor: Alon Kushnir, Givat Ada (IL)

(21) Appl. No.: 18/275,782

(22) PCT Filed: Feb. 6, 2022
(86) PCT No.: PCT/IL2022/050154

§ 371 Date: Aug. 3, 2023

(30) Foreign Application Priority Data
Feb. 8, 2021 (IL) 280733
Oct. 26, 2021 (IL) 287589

(51) Int. Cl. A61K 35/14; A61K 33/06; A61P 17/02

(52) U.S. Cl. CPC A61K 35/14; A61K 33/06; A61P 17/02

(57) ABSTRACT

The present disclosure provides a method and a kit for treating an abnormal hollowed space of a subject, such as a fistula, e.g. an anal fistula or a stoma space. For example, the abnormal hollowed space can be an anal fistula, a stoma opening, or a pilonidal sinus. The method utilizes autologous blood, which is derived from the subject, by introducing it into the abnormal hollowed space and let it clot within the abnormal hollowed space thereby forming a coagulated blood mass therein, namely a blood clot, that enhances the healing process of the abnormal hollowed space. The healing may be a complete or partial closure or reduction in the size or volume of said space; or the formation of healthy tissue within the former abnormal hollowed space.

### TECHNOLOGICAL FIELD

The present disclosure is in the field of medical treatment of abnormal hollowed space in a subject, in particular fistula or anal fistula.

### BACKGROUND ART

References considered to be relevant as background to the presently disclosed subject matter are listed below:
WO 2019/058373
WO 2019/058375
U.S. Pat. No. 9,180,142

Acknowledgement of the above references herein is not to be inferred as meaning that these are in any way relevant to the patentability of the presently disclosed subject matter.

### GENERAL DESCRIPTION

The present disclosure provides a method and a kit for treating an abnormal hollowed space of a subject, such as a fistula, e.g. an anal fistula or a stoma space. The term “abnormal hollowed space” in the context of the present disclosure should be interpreted as an abnormal cavity that is formed in human or nonhuman animal due to or in a result of a certain medical condition. For example, it can be an anal fistula, a stoma opening, or a pilonidal sinus. The method utilizes autologous blood, which is derived from the subject, by introducing it into the abnormal hollowed space and let it clot within the abnormal hollowed space thereby forming a coagulated blood mass therein, namely a blood clot, that enhances the healing process of the abnormal hollowed space. The healing may be a complete or partial closure or reduction in the size or volume of said space; or the formation of healthy tissue within the former abnormal hollowed space.

The abnormal hollowed space treated, in accordance with some embodiments of this disclosure, includes at least one opening at the external surface of the body. Namely, the hollowed space is accessible through an opening that is formed at a portion of the skin of the subject. By other embodiments, the abnormal hollowed space may open into an internal body cavity, for example may be a fistula between two tubular organs (e.g. between the colon and the urethra).

Thus, a first aspect of the present disclosure provides a method for treating an abnormal hollowed space of a subject. The method comprises: (i) obtaining or withdrawing whole blood from the subject. This may be performed by any known manner in the art. The method further includes (ii) mixing the subject's blood with one or more coagulation agents or anti-anticoagulation agents to thereby initiate clotting process of the blood. Then, (iii) prior to complete coagulation of the blood, introducing, e.g. injecting, the blood with the coagulation agent into said hollowed space and (iv) permitting the blood to coagulate in said abnormal hollowed space until a complete coagulation is obtained, and a clot is formed within the abnormal hollowed space. It is to be noted that when referring to the term “prior to complete coagulation of the blood” it means that the coagulation process is initiated but the blood is still in a flowable state and can flow, e.g. by injection, into the abnormal hollowed space and complete the coagulation therein, namely solidify to form a blood clot in the abnormal hollowed space. The clot may be maintained in the space into which it was injected for a defined period of time, e.g., hours or days, or maintained throughout the healing process. The coagulated blood mass within the abnormal hollowed space may be either absorbed or naturally degraded, may be naturally pushed out of the healing hollowed space or may be removed after a certain time period.

In some embodiments of the method, comprises withdrawing whole blood from the subject and mixing it with an anticoagulation agent prior to introduction of the blood into the hollow space to be treated. This allows a control of the coagulation process, e.g., fine-tuning of the coagulation time and the physical properties of the resulting coagulated blood by the nature of the anticoagulant or its concentration.

In some embodiments of the method, the anticoagulant is selected from a group consisting of: ACD-A (Anticoagulant Citrate Dextrose, Solution A), EDTA (ethylenediaminetetraacetic Acid), EGTA (ethylene glycol tetraacetic acid), a citrate, Heparin and oxalate.

In some embodiments, the method further comprises, in the instance that said hollowed space has two or more openings, e.g. a fistula, sealing one or more openings of the hollowed space, e.g., a distal opening of the fistula, thus creating a sinus, namely maintaining only one opening open. This step is carried out any time prior to (iii), above. In some embodiments, the method further comprises (iv) applying a medical dressing or a seal onto an opening of the hollowed space to prevent leakage of blood from the abnormal hollowed space or sinus, therefore ensuring its clotting therein.

In some embodiments, the method further comprises sealing one of the openings of the abnormal hollowed space by inflating an inflatable element to occlude one of the openings. For example, in the case of an annal fistula, such inflation of an inflatable element may be in the rectum; the walls of the inflatable element thereby sealing the fistula's opening into the rectal lumen. After the opening is sealed by the inflated element, the coagulating blood is introduced into the abnormal hollowed space through a non-occluded opening. The inflatable element may, in some cases, be maintained in situ for a limited time, e.g. after permitting the blood to coagulate within the abnormal hollowed space to a sufficient degree, the inflatable element may be deflated and retrieved. This is relevant, for example, in the case of anal fistula, wherein one of the openings of the anal fistula is in the wall of the rectal lumen, while the blood is introduced from the opening formed on the external skin near the annus. Thus, the inflatable element is introduced via the rectum in a non-inflatable state and once in position is inflated to the inflated state to seal the opening into the rectal lumen and only after such sealing the coagulating blood is introduced via the external opening. The blood is then allowed to be coagulated within the hollowed space of the anal fistula, e.g. for several minutes and the inflatable element may then be deflated and removed. Once the blood is sufficiently coagulated, the inflatable element may not be required to retain the coagulant within the anal fistula is deflated to the non-inflated state and retrieved via the rectum and the coagulated blood remains within the hollowed space of the anal fistula.

By some embodiments of the method, a medical dressing or a seal is applied onto an opening of the treated hollowed space, which may be immediately or after a time period following the introduction of the coagulating blood thereinto, e.g., after a period of between 3-15 minutes from said introducing, or after a period of between 1, 2, 3, 4, 5, 6, 7, 8 and 10, 11, 12, 13, 14, 15, 16 minutes, or about 9 minutes. It is to be noted that this period is typically the time frame for permitting the blood to coagulate within the abnormal hollowed space. In some embodiments, this time frame can be less than 10, 9, 8, 7, 6, 5, 4, 3, 2 or at times less than 1 minute.

The term “about” should be understood as a deviation of up to 20% from the nominated value. For example, a period of about 10 minutes is referred to any period of between 8-12 minutes.

In some embodiments of the method, the one or more coagulation agents are in a liquid or a powder form.

In some embodiments of the method, the one or more coagulation agents or anti-anticoagulating agents are selected from: Kaolin, Ca²⁺ (e.g. in the form of calcium salts such as Calcium Gluconate), Mg²⁺, negatively charged phospholipid (PL) and protamine sulfate.

In some embodiments of the method, (ii) comprises first mixing the blood with Calcium Gluconate followed by mixing the blood and Calcium Gluconate mixture with Kaolin.

In some embodiments of the method, said hollowed space is a fistula. In some embodiments of the method, said fistula is an anal fistula.

In some embodiments of the method, the hollowed space is a sinus.

In some embodiments of the method, the hollowed space is a pilonidal sinus.

In some embodiments of the method, the blood is introduced or injected into the abnormal hollowed space in defined injection profile. The injection profile comprises first injecting the blood to a distal end of the hollowed space until filling the entire space, namely until the blood fills said space up to its proximal end. This profile allows to prevent inflation of the abnormal hollowed space, e.g. the fistula, in case it is flexible and to ensure that the injected blood fills the entire volume of the abnormal hollowed space.

In some embodiments of the method, elements (ii) and (iii) of the method (defined above) are performed simultaneously. Namely, while introducing or injecting the blood into the abnormal hollowed space, the blood is mixed with one or more coagulation agents or anti-anticoagulation agents.

In some embodiments of the method, prior to (iii), the method further comprising sealing the opening of the abnormal hollowed space with a seal having an injection port, and then performing (iii) wherein the introducing is carried out via the injection port.

In some embodiments of the method, at least one of the openings of the abnormal hollowed space is formed at an external surface portion of the body of the subject. Thus, said at least one opening is accessible to the introduction of coagulating blood thereto without the need to penetrate the body. Depending on the site of the abnormal hollow space to be treated, the opening can be formed, for example, on a rectal portion, an abdominal portion, or any other exposed portion on the body of the subject.

In some embodiments of the method, said hollowed space is an abdominal space. The abdominal space can for as a result of a stoma or stoma reversal surgery and the blood can be introduced through the stoma opening that constitutes an opening of the abnormal hollowed space. In a stoma reversal surgery, the coagulating blood that is introduced into the abdominal space contacts the stoma opening and also the rejoined bowel ends that were separated during the stoma surgery. The blood is then coagulated on the tissues to be healed, i.e. the stoma opening and the attachment interface between the two rejoined bowel ends, and enhances the healing process.

Yet another aspect of the present disclosure provides a kit of parts or a system for use in the treatment an abnormal hollowed space or cavity of a subject. The kit or system comprising one or more blood withdrawal devices for blood withdrawal from the subject; one or more blood collection receptacles for receiving the blood withdrawn from the subject; a coagulation assembly configured for mixing of the withdrawn blood with one or more coagulation agents for initiating coagulation process of the withdrawn blood; and an applicator for introducing the coagulating blood into the hollowed space.

In some embodiments of the kit or system, the one or more collection receptacles are vacuum tubes.

In some embodiments of the kit or system, the coagulation assembly comprises an enclosure for receiving the withdrawn blood and mixing it with one or more coagulation agents or anti-anticoagulation agents.

In some embodiments, the kit further comprises one or more anticoagulation agents for mixing with the withdrawn blood to prevent its natural coagulation process and allow to controllably induce its coagulation at a desired time.

In some embodiments of the kit, the one or more anti-coagulation agents are comprised within said one or more blood collection receptacles.

In some embodiments of the kit, the anticoagulation agent is selected from a list consisting of: ACD-A (Anticoagulant Citrate Dextrose, Solution A), EDTA (ethylenediaminetetraacetic Acid), EGTA (ethylene glycol tetraacetic acid), a citrate, Heparin and oxalate.

In some embodiments of the kit, the one or more coagulation agents or anti-anticoagulating agents are selected from: Kaolin, Ca²⁺ (e.g. in the form of calcium salts such as Calcium Gluconate), Mg²⁺, negatively charged phospholipid (PL) and protamine sulfate.

In some embodiments, the kit further comprising a seal for sealing an opening of the abnormal hollowed space. The seal comprises an injection port for allowing injection of the coagulating blood into the abnormal hollowed space via the seal.

In some embodiments of the kit, the abnormal hollowed space is a fistula. In some embodiments of the kit, the fistula is an anal fistula.

In some embodiments of the kit, the abnormal hollowed space is a sinus.

It is to be noted that the term “sinus” refers to a sac or cavity in any organ or tissue, or an abnormal cavity or passage caused by the destruction of tissue.

In some embodiments, the kit is intended for use in a method for treating an abnormal hollowed space or cavity of a subject; the method comprising:
(i) withdrawing or obtaining whole blood from the subject;
(ii) mixing the subject's blood with one or more coagulation agents or anti-anticoagulation agents;
(iii) prior to complete coagulation of the blood, introducing the blood with the coagulation agent into said hollowed space; and
(iv) permitting the blood to coagulate in said space until a complete coagulation is obtained, and a clot is formed within the abnormal hollowed space.

In some embodiments, the kit is intended to be used in any of the above embodiments of the method.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-1D are schematic illustrations presenting different stages of a non-limiting example of the method according to an aspect of the present disclosure.

FIGS. 2A-2B are flow diagrams of non-limiting examples of different embodiments of the method according to an aspect of the present disclosure.

FIGS. 3A-3E are schematic illustrations presenting different stages of a procedure for treating an anal fistula.

### DETAILED DESCRIPTION

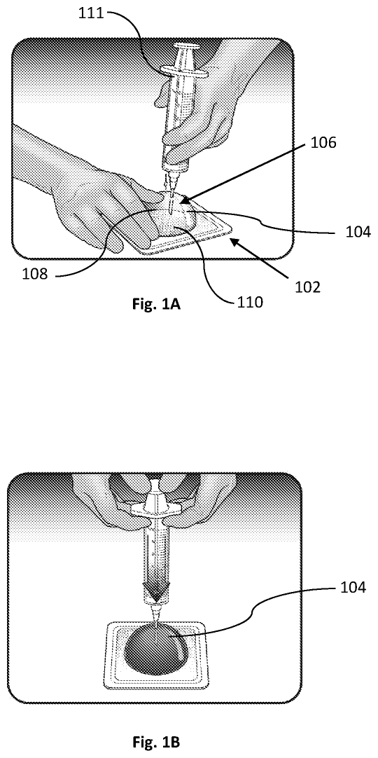

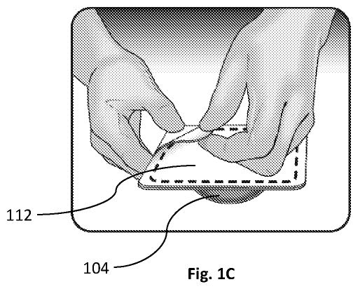

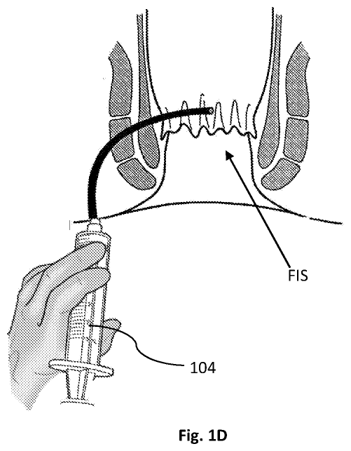

The method of the present disclosure is directed for treating abnormal hollowed space existing in a subject, e.g. anal fistula. The method first requires obtaining whole blood from the subject having an abnormal hollowed space to be treated. This is performed by withdrawing blood from the subject in any known method. After the blood is obtained, it is typically mixed with anti-coagulation agent for allowing to preserve the blood in a non-coagulated liquid form until it is desired to be coagulated to form a clot. Reference is now made to FIGS. 1A-1C, which are schematic illustrations presenting different stages of an embodiment of the method according to an aspect of the present disclosure, following the withdrawal of the blood from the patient. FIG. 1A shows a coagulation assembly 102 that includes a sealed blister 104 confining a sterile volume 106 for receiving drawn blood from the subject. The sealed blister 104 is formed of a blood injection portion 108, that can be transparent or semi-transparent to allow visualization of the process, and a coagulant-containing surface 110 that holds a liquid or powder form of one or more coagulating agents or anti-anticoagulating agents. By using a piercing element 111, e.g. a syringe with an adapted needle, the blood injection portion 108 is pierced at two different locations to form two openings, as can be seen in FIG. 1A, thereby allowing the introduction of blood into the sterile volume 106 from a first opening while air escapes from the second opening. The drawn blood is then injected into the sterile volume 106, as is exemplified in FIG. 1B, and the coagulating agents or anti-anticoagulating agents are mixed with the subject's blood to initiate the clotting process. The mixing can be enhanced by shaking the coagulation assembly 102, following sealing the openings in the blood injection portion 108 to prevent leakage of blood while shaking. The blister 104 is then unsealed, e.g. by removing the sealing base 112 at the bottom of the blister 104, as is exemplified in FIG. 1C, or by piercing the blood injection portion 108 to allow withdrawal of the coagulating blood into a syringe. The process then includes withdrawing the coagulating blood from the blister and then inject it into the abnormal hollowed space, e.g. fistula or into the sinus cavity in the instance that one or more of the openings of the abnormal hollowed space is sealed, e.g. by suturing procedure. This is exemplified in FIG. 1D, which shows blood with coagulating agents or anti-anticoagulating agents stored in a syringe 114 that is injected into a fistula FIS to permit its coagulation therein.

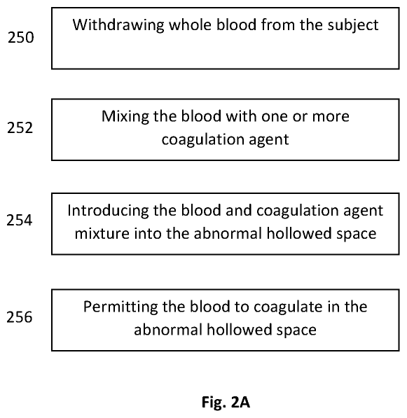

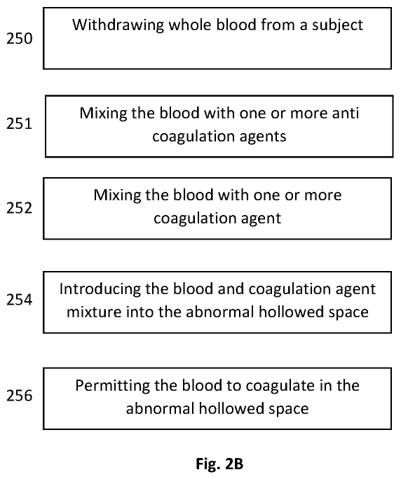

Reference is now being made to FIGS. 2A-2B, which are flow diagrams exemplifying different embodiments of a method according to an aspect of the present disclosure. The method being exemplified in FIG. 2A includes withdrawing whole blood from a subject 250 having an abnormal hollowed space to be treated, e.g. a fistula or an anal fistula. The method further includes mixing the withdrawn blood with one or more coagulation agents or anti-anticoagulation agents 252. Then, the method includes introducing the blood and coagulation agent mixture into the abnormal hollowed space 254. This is performed while the blood is still in a flowable form, namely prior to its complete coagulation. While the blood is in the hollowed space, the method includes permitting the blood to coagulate in the abnormal hollowed space 256, thereby forming a blood clot withing the abnormal hollowed space.

Reference is now being made to FIG. 2B, which differs from FIG. 2A by including after withdrawing whole blood from the subject 250 mixing the blood with one or more anti coagulation agents, thus inhibiting the natural clotting process of the blood and allowing to controllably induce the clotting process thereof at a desired timing.

It is to be noted that in the instance it is required to seal one or more openings of the abnormal hollowed space, the method further includes sealing one or more of the openings to create a sinus into which the clotting blood is introduced.

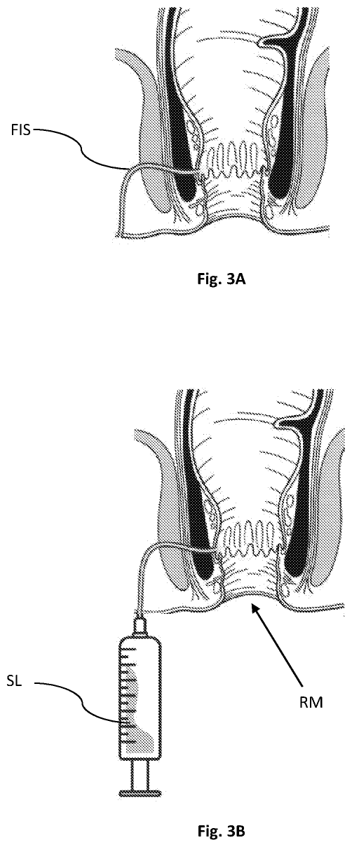

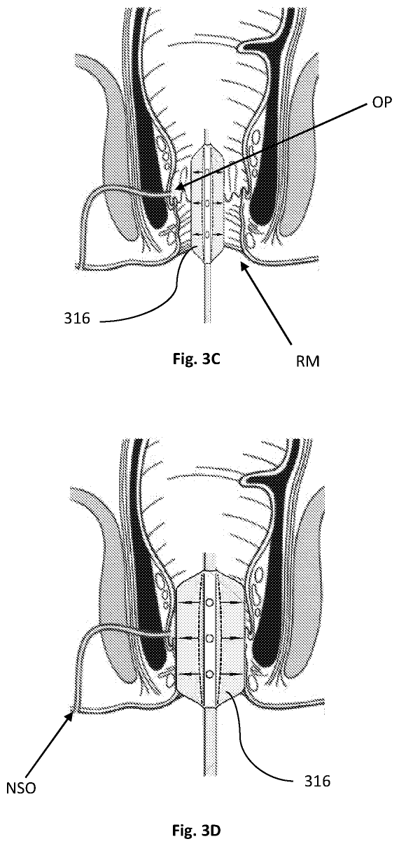

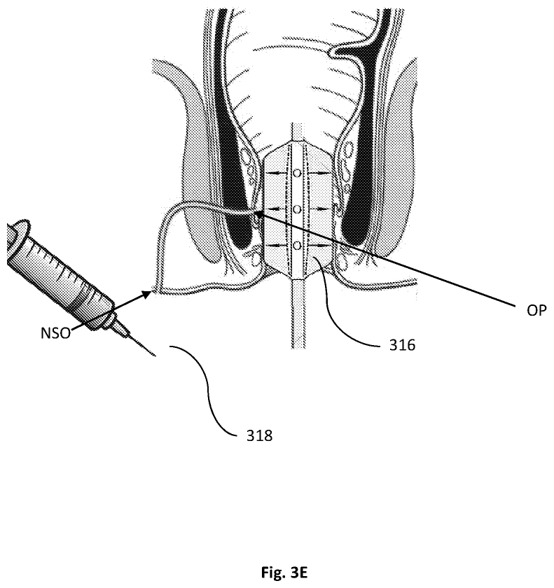

Reference is now being made to FIGS. 3A-3E, which are schematic illustrations exemplifying a procedure for treating anal fistula according to the present disclosure. FIG. 3A shows the initial pathology of the subject, which consists of an abnormal hollowed space formed by ab anal fistula FIS. The anal fistula FIS is first cleaned by introducing a sterilizing liquid SL into the anal fistula FIS from one opening thereof, as shown in FIG. 3B. Typically, the liquid is introduced via a first opening and discharged via a second opening of the anal fistula. Then, an inflatable/deflatable element 316 is introduced via the rectum RM when it is in its deflated state, as can be seen in FIG. 3C. Once positioned in the proximity of the opening OP that is accessible only via the rectum, the inflatable/deflatable element 316 is inflated to its inflated state, as shown in FIG. 3D, and coagulating whole blood 318 that was withdrawn from the subject is introduced via a non-sealed opening NSO of the fistula FIS. The whole blood is allowed to sufficiently coagulate within the fistula FIS, before the inflatable/deflatable element 316 is deflated to its deflated state and retrieved via the rectum RM.

### EXAMPLES

The following are examples of clinical trials based on various aspects of the present invention.

### Example 1—an Autologous Whole Blood Clot Treatment in Anal Fistula Patients

Purpose: An anal fistula is a chronic inflammatory process that doesn't usually heal spontaneously and surgery remains the definitive treatment. The high rates of both fistula recurrence and the potential risk of fecal incontinence continue to be major problems following the surgical procedures.

ActiGraft is an autologous blood clot, created from the patient's own peripheral blood forming a fibrin clot that promotes regeneration of cells via physical and biological mechanisms. ActiGraft was found to promote chronic wound healing from the stagnant inflammatory phase to the proliferative phase, resulting in cellular proliferation, wound bed granulation tissue formation and wound closure.

Methods: 29 patients with chronic anal fistulas underwent a surgical procedure including fistula cleaning and debridement, suturing of the internal, rectal side, opening. Water leak test was performed. Following the procedure, 18 mL of blood was drawn from the patient and was mixed with kaolin and calcium gluconate and immediately injected into the wound of the fistula tract, allowing the blood to coagulate inside by blocking the sinus. Fistula healing verification was done by visual assessment and an MRI at 3 months post-application.

Results: At the time of this report, 8 subjects completed the 6 months follow up, while all other 19 subjects are ongoing. An interim analysis was performed on the per protocol population showing that ActiGraft treatment resulted in a complete healing in 7 out of 8 patients, confirmed by an MRI, that have completed the 6 months follow-up. Interestingly, a Crohn's disease patient was among the 7 patients with confirmed healing, showing a dramatic effect of ActiGraft on this sub-population patients.

Conclusions: ActiGraft was found to be safe and effective in treating patients with anal fistula including a Crohn's disease patient, achieving a complete healing at 3 months post-surgery.

### Example 2—Safety and Efficacy of an Autologous Whole Blood Clot Treatment in Pilonidal Sinus Following Minimal Invasive Surgery

Purpose: Pilonidal disease is a common condition that affects young patients worldwide. It is most commonly arising in the hair follicles of the natal cleft of the sacrococcygeal, causing a foreign body reaction that leads to midline pit formation, a condition that may cause infection, significant discomfort, pain, immobility, and loss of work productivity in healthy young adults. The common treatment of pilonidal disease consists of surgical excision of the sinus tracts, associated with surgical risks.

ActiGraft, an autologous blood clot, was found to be safe and effective in healing cutaneous post-surgical and chronic wounds. A blood clot is the body's natural and optimal provisional wound matrix, serves as a provisional matrix which cells can migrate to, promoting the regeneration of localized tissue. Blood clot treatment in pilonidal sinus with minimal invasive-surgical procedure, serves not only as a provisional matrix, but as a regenerative tissue that can seal the sinus cavity and lead to the formation of new granulation tissue and re-epithelization, and thus reducing healing time and preventing sinus recurrence and complications.

Methods: 43 Patients with pilonidal sinus underwent a minimally invasive trephine surgery. immediately following the procedure, 18 mL of blood that was drawn and was mixed with Kaolin and calcium gluconate and immediately injected into the sinus cavity, allowing the blood to coagulate inside to create a blood clot. pilonidal sinus healing was validated visually 12 weeks post-surgery.

Results: An interim analysis on the per-protocol population was performed. 25 patients completed the 12 weeks follow-up, 13 patients are still in the follow-up phase, and a loss of follow-up occurred in 5 patients. Complete healing was confirmed in 21/25 patients that have completed the study with no reported adverse events.

Conclusions: ActiGraft was found to be safe and effective in reaching a complete closure of the sinus cavity following a minimal invasive trephine pilonidal sinus surgery.

ActiGraft contains a fibrin scaffold that holds damaged tissues together and provides the provisional matrix and factors required for the natural healing process. ActiGraft properties position it as a promising treatment in pilonidal sinus.

### Example 3—an Autologous Blood Clot Accelerate Wound Closure after Stoma Reversal

Purpose: Stoma reversal surgery may result in various complications which can increase the length of stay, significant healthcare-related costs, and poor long-term outcomes. SSI continues to be the most common complication ranging from 2% to 41% and therefore reducing the rate of SSI after SR is a significant unmet need.

An autologous whole blood clot creates at a point of care from the patient's own peripheral blood, creates a protective scaffold that promotes the wound healing process and increases cell granulation, and was suggested to delay infiltration of bacteria into the wound.

Objective: Four patients who underwent SR operation and autologous whole blood clot were reviewed. At the conclusion of surgery the wound was left partially opened and 18-24 hours post-operation, blood was drawn from the patient and was mixed with Kaolin and calcium coagulant and immediately injected into the open wound, allowing the blood to coagulate inside the wound to create a blood clot. The patients were followed for signs of infection and closure rate.

Outcomes: Autologous blood clot treatment was applied to 4 patients following SR. Complete wound closure was noticed 3 weeks after the autologous blood clot treatment in all patients with no signs of infection. The autologous blood clot treatment promotes wound healing process and may accelerate the closure of the SR wounds and the risk of infection.

1. A method for treating an abnormal hollowed space formed in a body portion of a subject, the method comprising:
(i) withdrawing whole blood from the subject;
(ii) mixing the subject's blood with one or more coagulation agents or anti-anticoagulation agents;
(iii) prior to complete coagulation of the blood, introducing the blood with the coagulation agent into said hollowed space; and
(iv) permitting the blood to coagulate in said abnormal hollowed space.

2. The method of claim 1, wherein (i) comprises withdrawing whole blood from the subject and mixing it with an anticoagulation agent.

3. The method of claim 1, wherein the anticoagulant is selected from a group consisting of: ACD-A (Anticoagulant Citrate Dextrose, Solution A), EDTA (ethylenediaminetetraacetic Acid), EGTA (ethylene glycol tetraacetic acid), a citrate, Heparin and oxalate, and wherein said one or more coagulation agents or anti-anticoagulating agents are selected from: Kaolin, Ca²⁺ (e.g. in the form of calcium salts such as Calcium Gluconate), Mg²⁺, negatively charged phospholipid (PL) and protamine sulfate.

4. The method of claim 1, comprising, where said hollowed space has two or more openings, sealing one or more openings of the hollowed space for the creation of sinus.

5. The method of claim 1, wherein (iv) comprises applying a medical dressing or a seal onto an opening of the hollowed space.

6-9. (canceled)

10. The method of claim 1, comprising, prior to (iii), sealing the abnormal hollowed space with a seal having an injection port, followed by (iii), wherein said introducing is via the injection port.

11. The method of claim 1, wherein said hollowed space comprises at least one opening that is formed on an external surface of the body.

12. The method of claim 1, wherein said hollowed space is anal fistula.

13. The method of claim 1, wherein said hollowed space is a sinus or a pilonidal sinus or a stoma space formed in a result of a stoma surgery.

14-16. (canceled)

17. The method of claim 1, wherein said abnormal hollowed space comprises two or more openings and the method further comprises, prior to (iii), bringing an inflatable/deflatable element in its deflated state adjacent to one of the openings and inflating it to seal the opening to thereby allow maintaining of the blood in the hollowed space when it is introduced thereto via a non-sealed opening and deflating the inflatable/deflatable element following sufficient coagulation of the blood within the hollowed space and retrieving the inflatable/deflatable element.

18. (canceled)

19. A kit for use in the treatment an abnormal hollowed space formed in a body portion of a subject, the kit comprising:
one or more blood withdrawal devices for allowing withdrawal of blood from the subject;
one or more blood collection receptacles for receiving the blood withdrawn from the subject;
a coagulation assembly configured for permitting mixture of the withdrawn blood with one or more coagulation agents for initiating coagulation process of the withdrawn blood; and
an applicator for introducing the incomplete coagulated blood into the hollowed space.

20. The kit of claim 19, wherein said one or more collection receptacles are vacuum tubes.

21. The kit of claim 19, wherein the coagulation assembly comprises a volume for introducing the withdrawn blood, said volume is contactable with one or more coagulation agents or anti-anticoagulation agents.

22. The kit of claim 19, comprising one or more anticoagulation agents for mixing with the withdrawn blood.

23. The kit of claim 22, wherein the one or more anti-coagulation agents are comprised within said one or more blood collection receptacles.

24. The kit of claim 22, wherein the anticoagulation agent is selected from a group consisting of: ACD-A (Anticoagulant Citrate Dextrose, Solution A), EDTA (ethylenediaminetetraacetic Acid), EGTA (ethylene glycol tetraacetic acid), a citrate, Heparin and oxalate; and wherein said one or more coagulation agents or anti-anticoagulating agents are selected from: Kaolin, Ca²⁺, Mg²⁺, negatively charged phospholipid (PL) and protamine sulfate.

25. (canceled)

26. The kit of claim 19, comprising a seal for sealing an opening of said abnormal hollowed space, said seal comprises an injection port for allowing injection of blood to the abnormal hollowed space via said seal.

27. The kit of claim 19, wherein said abnormal hollowed space is an anal fistula.

28. The kit of claim 19, wherein said abnormal hollowed space is a sinus or a stoma space.

29-30. (canceled)

31. The kit of claim 19, for use in a method for treating an abnormal hollowed space of a subject, the method comprising:
(i) withdrawing whole blood from the subject;
(ii) mixing the subject's blood with one or more coagulation agents or anti-anticoagulation agents;
(iii) prior to complete coagulation of the blood, introducing the blood with the coagulation agent into said hollowed space; and
(iv) permitting the blood to coagulate in said space.

32. (canceled)
